SEQUENCE LISTING (2 sequences)

SEQ ID NO: 1 - DNA, 886 bp, Pantoea sp.
ctgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgccacaccatgggagtgggttgcaaaagaagtaggtagcttaaccttcgggagggcgcttaccactttgtgattcatgactggggtgaagtcgtaacaaggtaaccaagggc

SEQ ID NO: 2 - DNA, 1515 bp, Enterobacteria sp.
gcccttagagtttgatcctggctcagattgaacgctggcggcaggcctaacacatgcaagtcgagcggcagcggaaagtagcttgctactttgccggcgagcggcggacgggtgagtaatgtctgggaaactgcctgatggagggggataactactggaaacggtagctaataccgcataacgtcgcaagaccaaagagggggaccttcgggcctcttgctatcagatgtgcccagrtgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgmtraggttaataacctarkcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccaagggc

(19) United States
(12) Patent Application Publication
(10) Pub. No.: US 2010/0184170 A1
(43) Pub. Date: Jul. 22, 2010

(54) Development of a Fermentative Enrichment Culture and Two Pure Isolates that Biotransform High Concentrations of Chloroform and Other Halomethanes

(75) Inventors: Huifeng Shan, Tucker, GA (US); David L. Freedman, Clemson, SC (US); Harry D. Kurtz, JR., Central, SC (US)

Correspondence Address: DORITY & MANNING, P.A., POST OFFICE BOX 1449, GREENVILLE, SC, 29602-1449, US

(73) Assignee: CLEMSON UNIVERSITY, Clemson, SC (US)

(21) Appl. No.: 12/688,324

(22) Filed: Jan. 15, 2010

Related U.S. Application Data
(60) Provisional application No. 61/145,247, filed on Jan. 16, 2009.

(51) Int. Cl. C12P 1/04; C12N 1/20; C12P 7/40

(52) U.S. Cl. 435/136; 435/252.1; 435/170

(57) ABSTRACT

Disclosed are fermentative enrichment cultures and two pure isolates there from for use in biotransformation of halomethanes. Disclosed bioaugmentation cultures are dominated by Enterobacteriaceae, and are implemented together with one or more electron donors such as corn syrup, glucose, and the like, and a corrinoid catalyst such as vitamin B₁₂. Disclosed cultures and implementation methods can be utilized to transform single halomethanes or mixtures of halomethanes at high rates to non-toxic end products being primarily carbon monoxide, carbon dioxide, and, organic acids.

### CROSS REFERENCE TO RELATED APPLICATION

The present application claims filing benefit of U.S. Provisional Patent Application Ser. No. 61/147,247 having a filing date of Jan. 16, 2009, which is incorporated herein by reference in its entirety.

### BACKGROUND

In spite of major recent advances in bioremediation of contaminated groundwater, significant challenges remain. One of these involves developing effective methods to bioremediate halogenated methanes, including carbon tetrachloride (CT), chloroform (CF), and trichlorofluoromethane (CFC-11). In the 2007 CERCLA Priority List of Hazardous Substances, CF ranks number 11, the third highest among chlorinated organics, after vinyl chloride (VC) and polychlorinated biphenyls. This is a reflection of how frequently CF is detected in groundwater and its high level of toxicity. Although not ranked as highly, CT is also on the list, and CFC-11 is often a co-contaminant with CT and CF. Among these halomethanes, CF is the focal point for evaluating the feasibility of bioremediation because it is very toxic to most obligate anaerobic prokaryotes. Hence, until a substantial decrease in CF is achieved, little biological activity is likely to occur on the other contaminants, e.g., chlorinated ethenes. Inhibitory effects of CT and CFC-11 on anaerobes at low concentrations have been reported as well.

Bioremediation is a preferred method for cleaning many hazardous wastes from sites of contamination. Bioremediation strategies include biostimulation and/or bioaugmentation. In general, biostimulation refers to modification of in-situ environment to stimulate indigenous microorganisms capable of bioremediation including addition of nutrients, electron donors and/or acceptors, etc. Bioaugmentation refers to bioremediation by means of introduction of cultured microorganisms to a site.

A halogen-containing contaminant that is targeted in a bioremediation process may serve as primary substrate (e.g., an electron acceptor) in the metabolic pathway of an organism used in the process (termed halorespiration). Alternatively, a contaminant may serve as a secondary substrate, and only be transformed cometabolically. For instance, bacteria of the genus Dehalococcoides have been utilized in bioremediation of sites contaminated with tetrachloroethene and trichloroethene as well as polychlorinated biphenyls (PCBs). In general, Dehalococcoides sp. utilize an anaerobic reductive dechlorination pathway (i.e., halorespiration) in which chlorinated hydrocarbons serve as electron acceptors, and molecular hydrogen, typically obtained indirectly via fermentation of organic substrates, serves as the electron donor. In contrast, Desulforomonas michiganensis, using lactate as the primary substrate, can degrade the same types of contaminants. At this point in time, it appears that anaerobic biotransformation of CT, CF and CFC-11 is strictly a cometabolic process. No cultures have been shown capable of using these compounds as growth-linked substrates.

Unfortunately, bacteria utilized in bioremediation of many contaminants are obligate anaerobes, which complicates handling and processing of the cultures during site clean up. In addition, most bioremediation cultures will be destroyed by the targeted compounds following a limited time of interaction. As such, bioremediation processes will often require multiple culture inoculations at a site in order to carry out the desired clean-up process. Moreover, due to the toxicity of the targeted compounds to the microbes used in the processes, biotransformation of high concentrations of toxins is often highly challenging.

Very limited attempts have been made to biotransform high concentrations of halogenated compounds. For instance, Becker and Freedman (Environ. Sci. Technol., 1994, 28, 1942-1949) were able to biotransform 260 mg/L of chloroform in an enrichment culture grown on dichloromethane (DCM) as the sole organic and energy source with the addition of supplemental cyanocobalamin. However, use of the culture of Becker and Freedman is not practical for in situ bioaugmentation because it requires dichloromethane as growth substrate and only grows well at 35° C. In addition, due to competitive inhibition, a common intrinsic restriction of cometabolic processes, presence and transformation of secondary substrates (e.g., CF) shut down growth of the cultures on primary substrates. This DCM-growing culture was no exception.

What is needed in the art is development of bioaugmentation cultures that can be utilized in treatment of sites contaminated with high concentrations of halogenated methanes. Growth of the bioaugmentation cultures while transformation and alteration to the pathway from reductive chlorination to formation of more ecologically benign materials such as carbon monoxide (CO), carbon dioxide (CO₂), and organic acids would be highly desirable.

### SUMMARY

Disclosed in one embodiment is a bioaugmentation culture comprising a facultative anaerobic bacteria of the genus Pantoea, the bioaugmentation culture being capable of biotransforming a halogenated methane. For example, the bioaugmentation culture can include one or more of the isolates DHM-1B and DHM-1T. In one embodiment, the bioaugmentation culture can include the enrichment culture DHM-1.

A bioaugmentation culture can include additional components as well as the bacteria. For instance, a culture can include one or more of a primary substrate such as corn syrup, a sulfide salt, a buffer, and a corrinoid catalyst, e.g., a cobalamin.

In one embodiment, a bioaugmentation culture can include a biomass concentration of between about 2 milligrams per liter and about 10 milligrams per liter.

Also disclosed is a method for biotransforming a halogenated methane. A method can include combining a bioaugmentation culture with one or more halogenated methanes and growing the bacteria of the bioaugmentation culture in the presence of the methane(s) under anaerobic conditions. For example, the bioaugmentation culture can be combined with the one or more halogenated methanes in situ.

A method can also include acclimatizing the facultative anaerobic bacteria of the bioaugmentation culture to at least one of the halogenated methanes prior to an in situ combination.

Beneficially, the products of a transformation can include primarily carbon monoxide (CO), carbon dioxide (CO₂), and at least one organic acid.

Halogenated methanes can include, for instance, chloroform, carbon tetrachloride, and trichlorofluoromethane. In one embodiment, chloroform can be biotransformed according to disclosed processes. For instance chloroform at an initial concentration of between about 500 milligrams per liter and about 2000 milligrams per liter can be biotransformed.

### BRIEF DESCRIPTION OF THE FIGURES

A full and enabling description of the presently disclosed subject matter, including the best mode thereof, to one of ordinary skill in the art, is set forth more particularly in the remainder of the specification, including reference to the accompanying Figures, in which:

FIG. 1 graphically illustrates the growth of a bioaugmentation culture as disclosed herein over time as indicated by increase in protein concentration with contemporaneous biotransformation of chloroform from an initial concentration of 500 mg/L; and

FIG. 2 graphically illustrates the biotransformation of a mixture of halogenated methanes at high initial concentrations utilizing a bioaugmentation culture as described herein.

### SEQUENCE LISTINGS

SEQ ID NO.:1 depicts the 16S ribosomal RNA gene of the isolate DHM-1B (GenBank Accession No. FJ745299).

SEQ ID NO.:2 depicts the 16S ribosomal RNA gene of the isolate DHM-1T (GenBank Accession No. FJ745300).

### DETAILED DESCRIPTION OF REPRESENTATIVE EMBODIMENTS

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made to the disclosed subject matter without departing from the scope or spirit of the disclosure. For instance, features illustrated or described as part of one embodiment, may be used with another embodiment to yield a still further embodiment.

In general, disclosed herein are bioaugmentation cultures for use in biotransformation of halomethanes. More specifically, disclosed fermentative cultures can be indigenous and are understood to be dominated by members of the family Enterobacteriaceae. One particular enrichment culture comprising multiple different bacteria formed and utilized as described herein has been designated DHM-1. The enrichment culture DHM-1 is understood to include species of the genera Pantoea as well as species of the genera Enterobacter and/or Citrobacter.

Also disclosed are two pure isolates of DHM-1 designated DHM-1B and DHM-1T that can function equally to or even better than a mixed culture. The isolates DHM-1B and DHM-1T are believed to be members of the Pantoea genus and are generally referred to as such throughout this disclosure. DHM-1B has been found to grow comparatively fast as compared to the growth rate of DHM-1T.

A bioaugmentation culture can include a mixture of facultative anaerobic species, but this is not a requirement of the disclosed subject matter and in other embodiments, a bioaugmentation culture can include a single facultative anaerobic species. In general, a bioaugmentation culture can include at least one of Pantoea spp. strain DHM-1B and DHM-1T.

In one embodiment, disclosed bioaugmentation cultures can be applied to a bioremediation site together with one or more electron donors and a corrinoid catalyst, which can allow disclosed cultures to take additional advantage of activities of halomethane transformation. Disclosed cultures and implementation methods can be utilized to transform single halomethanes or mixtures of halomethanes, optionally in the presence of other contaminants, and can transform high concentrations of the contaminants with products being primarily CO, CO₂ and one or more organic acids.

A mixed bioaugmentation culture can include bacteria of other species, in addition to species of DHM-1, e.g., Pantoea spp. For instance, a bioaugmentation culture can include Propionibacteriaceae sp., which has been utilized in petroleum bioremediation. Other bacteria as may be included in a bioaugmentation culture can include those that have been utilized in bioaugmentation cultures in the past, for instance, Dehalococcoides sp. and/or Desulforomonas sp., as discussed above, or other species as are known in the art, including, without limitation, Pseudomonas sp. (utilized for bioremediation of CT), Sphingomonas sp., Rhodococcus sp., Arthrobacter sp. (all three of which have been utilized for bioremediation of nitroaromatic and chlorinated pesticides), and the like. Preferred microorganisms included in any mixed culture can generally be determined according to site and/or process conditions such as, for example, specific contaminants at a site, aerobic or anaerobic conditions of inoculation and treatment, and so forth.

Beneficially, all members of the Enterobacteriaceae family are facultative anaerobes, which can greatly simplify transport, storage, and inoculation procedures using the cultures. Moreover, Enterobacteriaceae bacteria are not detrimentally affected by the presence of halomethanes under anaerobic conditions. More specifically, disclosed cultures can grow under either aerobic or fermentative conditions without losing the ability to anaerobically biotransform halomethanes such as CF. Thus, disclosed cultures are relatively easy to handle for in situ applications as compared to obligate anaerobes.

Biotransformation of halomethanes by disclosed cultures takes place in anaerobic conditions. Nonetheless, establishment of anaerobic conditions prior to bioaugmentation is not necessarily required because bioaugmentation with a disclosed fermentative culture together with electron donor can establish the low redox potential needed for the subsequent biotransformation in a short period of time, making its practical application much easier. Accordingly, in one embodiment, microorganisms of disclosed cultures can be acclimatized to high concentrations of halomethanes prior to a biotransformation process. It is believed that an acclimatization process prior to biotransformation can accelerate the biotransformation process. It should be understood, however, that an acclimatization process is not a requirement of disclosed methods.

While not wishing to be bound by any particular theory, it is believed that utilization of primary substrate (e.g., corn syrup) by Pantoea spp. takes place through an anaerobic fermentation process via a mixed organic acid pathway. Unlike chlororespiration of chlorinated ethenes, which is a metabolic process, halomethane contaminants are transformed cometabolically by disclosed cultures, and specifically, via substitutive and hydrolytic pathways rather than reductive dechlorination. Products of the bioremediation methods can be primarily CO, CO₂, and organic acids.

Beneficially, as the presence of halogenated compounds has no detrimental effect on the Enterobacteriaceae microorganisms, the initial biomass concentration can be quite low and still result in transformation of high concentrations of contaminants. For instance, the initial biomass concentration of a bioaugmentation culture can be as low as about 2 mg/L, even in the presence of extremely high levels of contaminants, e.g., greater than about 500 mg/L CF, and complete biotransformation of the contaminants can be carried out without addition of supplemental microorganisms. In one embodiment, the initial biomass concentration of a bioaugmentation culture can be between about 2 and about 10 mg/L.

In addition to one or more microorganisms, a bioaugmentation culture can also include a primary substrate. In general, any substrate that can function as an electron donor in the metabolic pathway of microorganisms of the culture can be utilized. According to one preferred embodiment, a primary substrate can be selected from easily biodegradable materials, so as to provide a more ecologically-friendly culture for inoculation at a clean up site. Suitable easily biodegradable substrates can include, without limitation, corn syrup, glucose, fructose, and the like.

A culture can include a primary substrate in an amount suitable to support the microorganisms contained therein, according to known systems and methods. For instance, a culture can initially include about 5 mM primary substrate, with appropriate supplementation thereafter as necessary.

Disclosed bioaugmentation cultures also include a corrinoid catalyst. More specifically, corrinoids encompassed in the disclosed subject matter include cobalamins including a central corrin ring containing four ligands for the central cobalt ion, a lower benzimodazole ligand, and an upper ligand that can vary depending upon the specific cobalamin. For instance, natural cobalamins encompassed by the present disclosure include adenosylcobalamin (AdoCbl), methylcobalamin (MeCbl), and hydroxocobalamin (OHCbl). Cyanocobalamin is a common industrially produced stable cobalamin form, and is a synthetic compound not found in nature. Cyanocobalamin can be utilized as a catalyst in one preferred embodiment of the present disclosure. While vitamin B-12 generally refers to the entire class of cobalamins, as utilized herein the terms ‘vitamin B₁₂’ or simply ‘B₁₂’ generally refer to the synthetic form cyanocobalamin.

In general, an initial bioaugmentation culture can include a cobalamin catalyst in a molar ratio of about 3% of halomethane concentration to be treated.

The addition of a corrinoid catalyst to a bioaugmentation culture can accelerate the rate of biotransformation of the halomethane contaminants and can also shift the biotransformation process from a reductive dechlorination process to a process that favors substitutive and for hydrolytic reactions. As such, the daughter products of a biotransformation process can primarily include CO₂, CO, and organic acids, with no further biotransformation processes necessary to decontaminate a site. For example, the major products of a biotransformation process as disclosed herein can include about 70% CO and CO₂ combined, followed by organic acids (e.g., formic acid, acetic acid, propionic acid, etc.), and minor amounts of carbon disulfide.

Unlike reductive dechlorination of chlorinated ethenes, where complete dechlorination is achievable, reductive dechlorination of CT to CF is often a significant pathway, but CF to dichloromethane (DCM) never completes, and further reduction to chloromethane and methane is minor. Therefore, the reductive dechlorination pathway is not sufficient to transform halomethanes to non-toxic end products. In order to completely clean a site, these reduced halomethanes require another, different bioremediation process. Through utilization of the presently disclosed bioaugmentation cultures, multiple types of processing are not required, as the disclosed cultures can biotransform single or mixed halomethane compositions to acceptable products in a single biotransformation process.

Disclosed bioaugmentation cultures can transform high concentrations of halomethanes. For instance, a bioaugmentation culture including a single DHM-1B or DHM-1T isolate can remediate an initial CF concentration of up to about 500 mg/L, and the enrichment culture DHM-1 can remediate an initial CF concentration of up to about 2000 mg/L. Moreover, at very high CF concentration, about 4,000 mg/L CF, growth of disclosed bioaugmentation culture DHM-1 is not adversely affected. At such extremely high CF concentrations, which is about 50% of the highest possible aqueous CF concentration, disclosed DHM-1 culture may not transform CF, but it can continue to grow on a suitable substrate (e.g., corn syrup).

Disclosed methods can also transform halomethanes at high rates. For example, according to disclosed methods, CF can be transformed by enrichment culture DHM-1 or by the isolates DHM-1B or DHM-1T at a rate as high as about 22 mg/L/d.

Other halomethanes can likewise be transformed at high starting concentrations by use of disclosed cultures. For instance, disclosed cultures can be utilized for bioremediation of CT in concentrations greater than about 15 mg/L, at rates of greater than about 1 mg/L/d; CFC-11 can be transformed in concentrations greater than about 30 mg/L at rates of greater than about 0.5 mg/L/d. Moreover, disclosed bioaugmentation cultures can be utilized to successfully remediate mixtures of halomethane contaminants.

Prior to application of a bioaugmentation culture as disclosed herein, it may be preferred to modify in-situ conditions by additional additives as are known in the art. Specific additional additives as may be desired can generally be determined based upon the specific characteristics of a treatment process and/or a clean up site, such as pH, nutrient content, redox potential, etc.).

For example, contaminated sites may include suitable salt content and nutrients in natural aquifers for biotransformation processes. However, in some embodiments, it may be desirable to provide limiting nutrients in a bioaugmentation culture.

According to one embodiment, the presence of sulfide can be provided through addition of one or more sulfide salts in the culture. For instance, the rate of CF biotransformation can be increased in the presence of sulfide, for instance about 0.5 mM sulfide included as a component of a modified salts medium in the culture. While the presence of sulfide in the culture is not required, the rate of CF biotransformation can be three to four times greater in the presence of sulfide. While not wishing to be bound by any particular theory, it is believed that a sulfide can be substituted by another reductant, e.g., zero valent iron (ZVI), in a biotransformation process.

A pH neutral environment is generally preferred during disclosed biotransformation processes. Accordingly, it may be desirable to incorporate a buffer in a bioaugmentation culture in those instances when contaminated soil is particularly acidic or basic.

Applications for utilization of disclosed bioaugmentation cultures are known to those of ordinary skill in the art. For instance, a bioaugmentation culture can be injected into a subsurface reclamation site. For example, an aqueous culture can be injected under low pressure, which can increase disbursement of the injected composition away from the injection sites. A culture can be injected through the end of a direct push rod, through temporary direct-push wells, or through temporary or permanent conventionally-drilled wells. A culture can also be injected using pneumatic or hydraulic fracturing according to known methods.

A number of manufacturers are generally known as can provide equipment for any desired in situ application process. For example, direct-push equipment is readily available that can be utilized for installation of temporary 1-inch direct-push wells or direct injection of through probe rods. By way of example, Geoprobe™ manufactures and sells tooling as may be utilized for injection of disclosed bioaugmentation cultures. The Geoprobe™ Pressure-Activated Injection Probe can be utilized with either 1.5-inch or 1.25-inch probe rods for “top-down” or “bottom-up” injection. Also available from Geoprobe™ are injection Pull Caps that provide a means to form a sealed connection to probe rods for injection while retracting the probe rod. An alternative method is to inject a bioaugmentation culture via a “bottom-up” approach, for instance by use of a rod including an expendable drive point tip.

The selection of temporary versus permanent injection points can generally depend upon site-specific conditions including: depth to water, drilling costs, flow rate per injection point and volume of fluid that must be injected. Injection designs can generally be optimized to provide the maximum injection flow rate while minimizing drilling costs.

In lower permeability formations, hydraulic and pneumatic fracturing can be used to enhance distribution of a bioaugmentation culture away from the injection point. Hydraulic fractures are formed when a fluid is pumped down a well at high pressures for short periods of time (hours) to create enough downhole pressure to crack or fracture the formation. For instance, water, optionally including high viscosity fluid additives, can be used as the high pressure fluid. To keep the fractures from closing when the pumping pressure is released, a propping agent such as sand or other coarse particulate material can be pumped into the formation, thereby creating a plane of high-permeability sand through which fluids can flow. The propagant remains in place once the hydraulic pressure is removed. This allows the fracture to remain open and enhances flow in the subsurface.

For pneumatic fracturing, a gas is pumped down a well at high pressures for short periods of time (hours) to create enough downhole pressure to crack or fracture the formation. The gas is injected into the subsurface at pressures that exceed the natural in situ pressures present in the soil/rock interface and at flow volumes exceeding the natural permeability of the subsurface.

Disclosed bioaugmentation cultures can be utilized in a variety of configurations in the subsurface, including source area treatments, plume treatments, and permeable reactive barrier (PRB) configurations. Source area and plume treatments involve distributing an initial bioaugmentation culture and any supplemental materials (e.g., supplemental primary substrate and/or supplemental cobalamin catalyst) in a portion of the source area or plume to degrade contaminants and/or reduce their mobility. A PRB can be formed by distributing a bioaugmentation culture in a line generally perpendicular to groundwater flow. As groundwater passes through the PRB, bioremediation of the contaminants can be carried out.

According to one embodiment, following injection, a bioaugmentation culture can provided desired remediation without further supplemental additives. According to another embodiment, a culture can be supplemented, with any or all components of the initially supplied bioaugmentation culture including individual microorganisms, primary or secondary substrates, catalysts, and so forth. Moreover, supplementation can be carried out at the injection points for the initial culture, or downstream thereof, as desired.

As discussed above, disclosed bioaugmentation cultures can exhibit an ability to biotransform extremely high concentrations of halomethanes, and specifically CF. This ability presents the possibility of using disclosed cultures for source zone remediation processes. For example, physical remediation strategies that target a nonaqueous phase liquid can precede biotic transformation of residual CF at very high concentrations, e.g., up to about 2000 mg/L.

The present disclosure may be better understood with reference to the following examples.

### Example 1

An enrichment culture, designated DHM-1, was developed by adding a soil sample from an uncontaminated area of a former industrial site in California to an anaerobic mineral salts medium (MSM) along with corn syrup (5 mM)+B12 (3% molar ratio to the CF). The contamination levels at other areas of the site include CF at concentrations as high as 500 mg/L, CT up to 10 mg/L, CFC-11 up to 26 mg/L, and 1,1-dichloroethene up to 10 mg/L. 1,1-Dichloroethene (DCE) is also present (up to 9.1 mg/L), presumably from dehydrohalogenation of 1,1,1-trichloroethane. Geochemical conditions that are unfavorable to anaerobic bioremediation (e.g., acidic pH, high halomethanes concentrations, and positive Eh levels) have existed at the site for decades.

After gradually acclimating the culture to 500 mg/L of CF, a 1% sequential transfer was made to MSM+corn syrup+B₁₂. The resulting enrichment culture biodegrades 500 mg/L of CF at an average rate of 16 mg/L/d or higher. The MSM is homologous to regular anaerobic media and contains 0.05 g/L yeast extract and 0.5 mM sulfide.

The presence of CF was also found to not inhibit growth of the bioaugmentation culture, based upon comparison of protein yields in cultures with no CF present and with 500 mg/L of CF added.

Utilizing ¹⁴C-labeled CT and CF, the main products of the biotransformation process were identified as CO, CO₂, and solubles (organic acids), as well as a minor amount of CS₂ from CT. In addition, dichloromethane and chloromethane amounts were insignificant. Results are shown in Table 1, below. Dichlorofluoromethane and chlorofluoromethane were detected from biotransformation of CFC-11, although not in stoichiometric amounts (not shown).

TABLE 1

| Compound | % of [¹⁴C]CF | % of [¹⁴C]CT |
|---|---|---|
| CH₄ | 0.5 | 0.1 |
| CM | 0.0 | 0.3 |
| DCM | 0.4 | 0.0 |
| CS₂ | 0.0 | 5.2 |
| CO | 67 | 14 |
| CO₂ | 2.7 | 51 |
| Solubles | 15 | 27 |
| Losses | 13 | 0.0 |

The microbes present in the enrichment culture were evaluated using denaturing gradient gel electrophoresis and sequencing of well separated bands. Enterobacter species predominate.

### Example 2

The isolates DHM-1B and DHM-1T were isolated via enrichment culture from the subsurface environment as soil cores from the location described in Example 1, above. Enrichment cultures were initiated using sediment from the saturated zone of these cores. Microcosm cultures were established under anaerobic conditions and monitored for removal of halocarbons. Changes in microbiota were monitored using denaturing gradient gel electrophoresis (DGGE), which allowed identification of the relevant organisms molecularly. Upon development of a microcosm culture that removed high levels of halomethane, cells were cultured on a standard laboratory medium under anaerobic conditions. Pure culture isolates were subsequently tested to show removal of halomethanes and it was determined that two distinct isolates, DHM-1B and DHM-1T, were competent at this process.

Specifically, samples of enrichment culture DHM-1 were streaked on Tryptic soy agar (TSA) and incubated (35° C.) aerobically for 24 h. Well isolated colonies were returned to mineral salts medium (MSM)+corn syrup+CF+B12 and incubated anaerobically. When CF transformation was complete, the process was repeated (i.e., restreaking, aerobic growth on TSA, and return of isolated colonies to liquid culture under anaerobic conditions). Samples from the resulting bottles were again restreaked and grown on TSA for up to 48 h. At this point, two morphologies were identified; one was a larger white (slightly yellow) colony (1-2 mm) that grew more quickly (i.e., within 24 h), the other a smaller yellow colony (about 1 mm) that grew more slowly (i.e., within 48 h). Samples of both colonies were restreaked on TSA four to seven times before returning the isolates to MSM with CF. The purity of the isolates was confirmed based on microscopic observation and sequencing of their 16S rRNA genes (provided herein as SEQ ID NO.:1 for DHM-1B and SEQ ID NO.:2 for DHM-1T). Both isolates demonstrated comparable CF-transforming capability to the enrichment culture DHM-1.

### Example 3

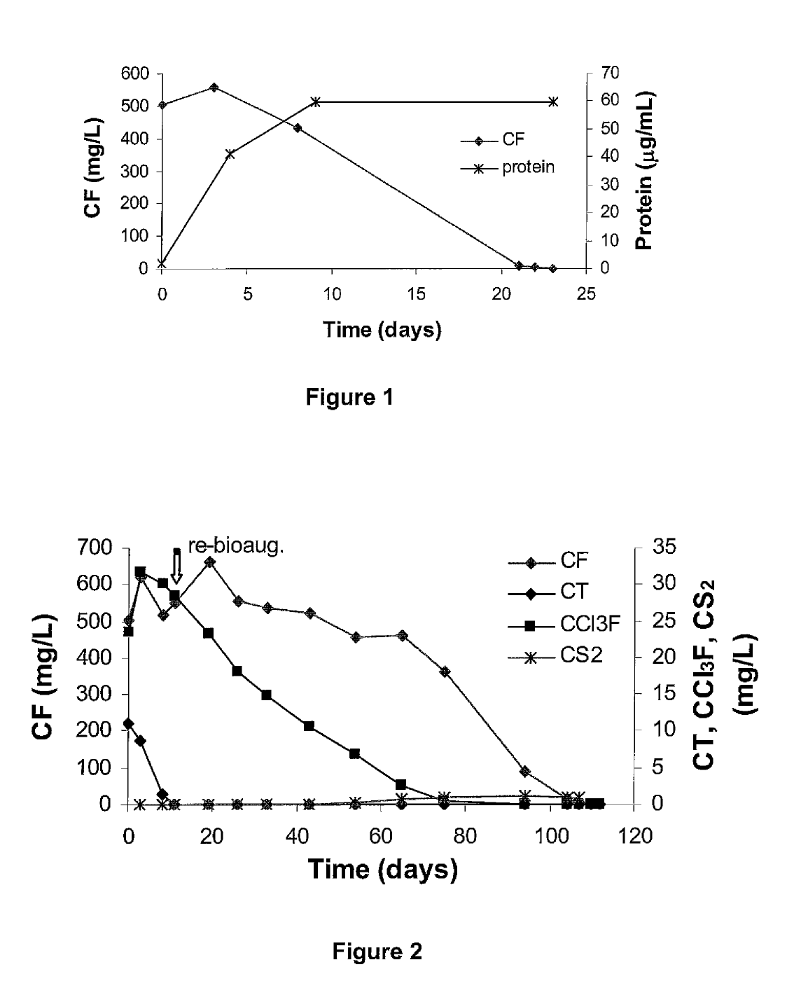

A bioaugmentation culture as described above in Example 1 was utilized to completely transform over 500 mg/L CF to non-toxic end products in 23 days at a rate of 22 mg/L/d (FIG. 1).

Absence of activity in live culture without B₁₂ and an abiotic control (media and B₁₂) confirmed the importance of supplementation with a catalytic amount of B₁₂ and also confirmed that the biotransformation was a biotic process.

Formation of reductive dechlorination products such as dichloromethane (DCM) and chloromethane, and carbon disulfide (CS₂) was negligible. ¹⁴C analyses revealed that carbon monoxide (CO) was the dominating end product, followed by organic acids such as acetate (Table 1). Furthermore, growth of the bioaugmentation culture was not adversely affect by either the presence or transformation of CF. As can be seen with reference to FIG. 1, significant growth of the culture was achieved in the first few days (within the lag phase of CF transformation) and maximum growth on a single dose of 5 mM corn syrup was reached approximately by day 9 while about 20% of CF was transformed. This growth pattern is similar to that under a scenario with microorganisms and corn syrup only (i.e., without CF and B₁₂).

### Example 4

In addition to transforming only CF, disclosed bioaugmentation cultures were examined for efficacy in transformation of mixtures of halomethanes including carbon tetrachloride (CT) and trichlorofluoromethane (CFC-11) as well as CF. A bioaugmentation culture as described in Example 1 was utilized. Sequential biotransformation of halomethanes occurred in the order of CT (11 mg/L), CFC-11 (24 mg/L) and finally CF (504 mg/L) (FIG. 2). Tailored B₁₂ addition and an extra dose of bioaugmentation at the end of CT transformation were found to be beneficial. CT was fully removed by day 10. Transformation of CFC-11 accelerated once CT was nearly gone. Likewise, transformation of CF did not start until concentration of CFC-11 dropped to low ppm level. Then, complete biotransformation was achieved by day 112. Formation of reductive dechlorination products such as DCM, CH₂ClF, and CS₂ was negligible.

It will be appreciated that the foregoing examples, given for purposes of illustration, are not to be construed as limiting the scope of this disclosure. Although only a few exemplary embodiments have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure as defined in the following claims and all equivalents thereto. Further, it is recognized that many embodiments may be conceived that do not achieve all of the advantages of some embodiments, yet the absence of a particular advantage shall not be construed to necessarily mean that such an embodiment is outside the scope of the presently disclosed subject matter.

What is claimed is:

1. A bioaugmentation culture comprising a facultative anaerobic bacteria of the genus Pantoea, the bioaugmentation culture being capable of biotransforming a halogenated methane.

2. The bioaugmentation culture of claim 1, further comprising one or more additional bacteria.

3. The bioaugmentation culture of claim 2, wherein the additional bacteria is of the genera Pantoea, Enterobacter, or Citrobacter.

4. The bioaugmentation culture of claim 2, wherein the bioaugmentation culture comprises DHM-1.

5. The bioaugmentation culture of claim 1, wherein the facultative anaerobic bacteria of the genus Pantoea is DHM-1B.

6. The bioaugmentation culture of claim 1, wherein the facultative anaerobic bacteria of the genus Pantoea is DHM-1T.

7. The bioaugmentation culture of claim 1, further comprising a primary substrate for the bacteria.

8. The bioaugmentation culture of claim 7, wherein the primary substrate is corn syrup.

9. The bioaugmentation culture of claim 1, further comprising a corrinoid catalyst.

10. The bioaugmentation culture of claim 9, wherein the corrinoid catalyst is a cobalamin.

11. The bioaugmentation culture of claim 1, further comprising a sulfide salt.

12. The bioaugmentation culture of claim 1, further comprising a buffer.

13. The bioaugmentation culture of claim 1, comprising a biomass concentration of between about 2 milligrams per liter and about 10 milligrams per liter.

14. A method of biotransforming a halogenated methane comprising:
combining a bioaugmentation culture with one or more halogenated methanes, the bioaugmentation culture comprising a facultative anaerobic bacteria of the genus Pantoea;
growing the bacteria of the bioaugmentation culture in the presence of the one or more halogenated methanes under anaerobic conditions; wherein
the one or more halogenated methanes are biotransformed by the facultative anaerobic bacteria during the growth process.

15. The method according to claim 14, wherein the bioaugmentation culture is combined with the one or more halogenated methanes in situ, the method further comprising acclimatizing the facultative anaerobic bacteria of the bioaugmentation culture to at least one of the halogenated methanes prior to the in situ combination.

16. The method according to claim 14, wherein the one or more halogenated methanes are biotransformed cometabolically.

17. The method according to claim 16, wherein the one or more halogenated methanes are biotransformed via substitutive and hydrolytic pathways.

18. The method according to claim 14, wherein products of the transformation comprise carbon monoxide, carbon dioxide, and at least one organic acid.

19. The method according to claim 14, wherein the one or more halogenated methanes comprises chloroform, carbon tetrachloride, or trichlorofluoromethane.

20. The method according to claim 19, wherein the one or more halogenated methanes comprises chloroform, and the chloroform is at an initial concentration of between about 500 milligrams per liter and about 2000 milligrams per liter.

21. The method according to claim 14, further comprising injecting the culture into a subsurface site.

22. The method according to claim 14, further comprising supplementing the bioaugmentation culture following the step of combining the bioaugmentation culture with one or more halogenated methanes.

23. The method according to claim 22, wherein the bioaugmentation culture is supplemented with a primary substrate or a catalyst.
